(19) United States
(12) Patent Application Publication
(10) Pub. No.: US 2007/0270382 A1
(43) Pub. Date: Nov. 22, 2007

(54) Composition for Protecting Skin

(76) Inventors: Changseo Park, Gyonggi-do (KR); Sunki Kim, Seoul (KR); Jinwook Kim, Gyonggi-do (KR); Hyeongjoon Kang, Gyonggi-do (KR); Bokyung Han, Seoul (KR); Younggon Lee, Gyonggi-do (KR); Soyoung Chung, Daejeon-city (KR); Wangkeun Choi, Gyonggi-do (KR); Sang June Nam, Gyonggi-do (KR)

Correspondence Address: MCDERMOTT WILL & EMERY LLP, 600 13TH STREET, N.W., WASHINGTON, DC, 20005-3096, US

(21) Appl. No.: 10/574,275

(22) PCT Filed: Oct. 1, 2004
(86) PCT No.: PCT/KR04/02517

§ 371 Date: Apr. 20, 2007

(30) Foreign Application Priority Data
Oct. 2, 2003 (KR) 10-2003-0068932
Sep. 22, 2004 (KR) 10-2004-0075864

(51) Int. Cl. A61K 31/664; A61P 17/00

(52) U.S. Cl. 514/78

(57) ABSTRACT

Disclosed are a composition for protecting skin and uses thereof. The composition comprises sphingomyelin as an active ingredient and protects the skin by inhibiting skin aging, treating a skin wound and improving a skin barrier.

### TECHNICAL FIELD

The present invention relates to a composition for protecting skin, and particularly to a composition for improving a skin barrier function, inhibiting skin aging and treating a skin wound. More specifically, the invention relates to a composition containing sphingomyelin.

### BACKGROUND ART

Sphingomyelin is a kind of sphingolipids and expressed as a following formula.

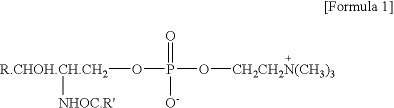

Sphingomyelin is the most plentiful lipid of lipid ingredients constituting a cell membrane, together with phospholipid, and occupies about 50% of the cell membrane in some tissues. Sphingomyelin occupies about 10% of the total lipids in brain tissues. It is also known that sphingomyelin is most present in erythrocytes by replacing phosphatidylcholine. Sphingomyelin is present not in a plant or microbe but in an animal only. Long chain bases constituting sphingomyelin are mainly sphingosine and sphinganine. Usually, long chain saturated fatty acids or unsaturated fatty acids having one double bond are mainly present as fatty acids. As can be seen from Tables 1 and 2, sphingomyelins are very various according to kinds of basic structures (sphingosine, sphinganine, phytosphingosine, etc.) constituting sphingomyelin and kinds of fatty acids bonded to sphingomyelin, and have very diverse distributions according to cells and tissues present in the human body. These also suggest that degradation products of sphingomyelin are diversely present and sphingomyelin can act as an intermediary of various signal transductions. Table 1 shows fatty acids constituting sphingomyelin by weight percent Ramstedt, B., Leppimaki, P., Axberg, M. and Slotte, J. P., “Analysis of natural and synthetic sphingomyelins using high-performance thin-layer chromatography”, Eur. J. Biochem., 266, 997-1002 (1999)).

TABLE 1

| | Fatty acids | | | | | | | | | |
|---|---|---|---|---|---|---|---|---|---|---|
| | 16:0 | 18:0 | 18:1 | 20:0 | 22:0 | 22:1 | 23:0 | 23:1 | 24:0 | 24:1 |
| Egg | 66 | 10 | 1 | 4 | 6 | 1 | 2 | | 5 | 3 |
| Brain of cattle | 3 | 42 | | 6 | 7 | 3 | 3 | 3 | 6 | 27 |
| Milk | 14 | 3 | 1 | 1 | 22 | | 32 | | 15 | 5 |

Table 2 shows long chain bases constituting sphingomyelin by weight percent (Ramstedt, B. et al., Eur. J. Biochem., 266, 997-1002 (1999)).

TABLE 2

| | Sphingoid bases | | | | | | |
|---|---|---|---|---|---|---|---|
| | d16:0 | d17:0 | d17:1 | d17:1-methyl | d18:0 | d18:1 | d19:0 |
| Egg | | | | | 7 | 93 | |
| brain of cattle | | | | | 19 | 81 | |
| Milk | 9 | 15 | 8 | 11 | 10 | 44 | 3 |

*d: dihydroxy base

It has been recently known that sphingomyelin and cholesterol are together present in peculiar sub-domains referred to as rafts. When one lipid of them decreases, another lipid is decreased together. Accordingly, it is interpreted that sphingomyelin plays an important role in regulating a cholesterol absorbing ability of a cell membrane.

Since sphingomyelin has mainly long chain saturated acyl chains, it has a higher melting point than that of glycerophospholipid, so that it can constitute a more rigid cell membrane. Such a rigid acyl chain is essential for construction of rafts and packing facilities, which are different from each other between sphingolipid and phospholipid, provide physical characteristics important for making a phase separation of the cell membrane. This constitutes sphingolipid-rich rafts (‘liquid-ordered’ phase) and glycerophospholipid-rich domains (‘liquid-disordered’ phase) surrounding the sphingolipid-rich rafts. The sphingolipid-rich rafts exhibit a relatively high resistance to surfactant and form rafts having a relatively small size (having a diameter of 50 nm and consisting of about 3,000 sphingomyelin molecules). Interactions between such rafts and diverse proteins in a cell have an important meaning regarding a signal transduction mechanism in the cell.

Generally, aging can be classified into photoaging and natural aging. The natural aging is structural and functional metabolic changes of skin according to degenerative changes of the body. Generally, as the skin becomes dry and thin and the generation of collagen is decreased, a wrinkle occurs and the skin loses its elasticity. In addition, abnormal blood vessels are developed and pigmentation such as dark spots increases. The photoaging causes damages of collagen and elastic fibers of the skin. As degrees of exposure to the sunlight are increased, amounts of wrinkles are proportionally increased and generation of proteolytic enzymes decomposing connective tissues, which are located below collagen and dermis, is also increased, so that the skin is severely injured.

### SUMMARY OF THE INVENTION

Accordingly, the present invention has been, made to solve the above-mentioned problems occurring in the prior art. The object of the present invention is to protect the skin from the sunlight and to rapidly repair physical damages of the skin. The other object of the invention is to prevent the skin from being dry and thinned due to the aging and losing its elasticity and the occurrence of wrinkles due to a decrease of generation of collagen. In addition, the other object of the invention is to protect the skin by improving a skin barrier function. Additionally, the invention has an object of curing a skin wound.

In order to accomplish the above objects, a composition for protecting skin comprising sphingomyelin as an active ingredient is provided.

According to the invention, the composition for protecting skin may be used for protecting the skin by inhibiting a skin aging.

According to the invention, the composition for protecting skin may be used for inhibiting a photoaging.

According to the invention, the composition for protecting skin may be used for protecting the skin by curing a wound.

According to the invention, the composition for protecting skin may be protecting for the skin by improving a skin barrier.

According to the invention, the composition for protecting skin may be used for rapidly repairing a damaged skin barrier.

According to the invention, the composition for protecting skin may be used for curing atopic skin.

According to the invention, the composition for protecting skin may be used for improving a wrinkle, itchy skin and softness of skin or preventing rough and scaly skin from occurring.

According to the invention, the sphingomyelin may be derived from milk or an egg.

According to the invention, the sphingomyelin may be hydrogenated sphingomyelin.

According to the invention, the composition for protecting skin may be a composition for oral administration or topical application.

### BRIEF DESCRIPTION OF THE DRAWINGS

The above and other objects, features and advantages of the present invention will be more apparent from the following detailed description taken in conjunction with the accompanying drawings, in which:

FIG. 1 is a graph showing a photoaging inhibitory effect of sphingomyelin;

FIGS. 2 to 4 are photographs showing a photoaging inhibitory effect of sphingomyelin, wherein FIG. 2 is a photograph showing a rat's state of skin in the case of UV irradiation+SM (sphingomyelin) application, FIG. 3 is a photograph a rat's state of skin in the case of UV irradiation+Vehicle application, and FIG. 4 is a photograph a rat's state of skin when only UV is irradiated;

FIG. 5 is a photograph showing a result of skin histological test after UV irradiation, for the purpose of illustrating a photoaging inhibitory effect of sphingomyelin;

FIG. 6 is a photograph showing an early stage of a test for examining a wound repairing effect of sphingomyelin, FIG. 7 is a photograph showing a state after 7 days from a treatment, and FIG. 8 is a photograph showing a state after 11 days from the treatment;

FIGS. 9 and 10 illustrate a result of test for demonstrating the wound repairing effect of hydrogenated sphingomyelin, wherein FIG. 9 is a photograph showing a state after 7 days from a treatment, and FIG. 10 is a photograph showing a state after 11 days from the treatment;

FIG. 11 illustrates a result of test for demonstrating an improvement effect of a skin barrier function of sphingomyelin, and is a graph showing variations of TEWL (Transepidermal water loss) average values when not taking sphingomyelin and when taking sphingomyelin, after injuring sebum film of the skin;

FIG. 12 is a graph showing the values of persons whose TEWL is particularly high;

FIG. 13 is a graph showing the values of persons whose skin barrier function is weak; and

FIG. 14 is a photograph showing a skin wrinkle improving effect of sphingomyelin.

### DETAILED DESCRIPTION OF THE PREFERRED EMBODIMENTS

Hereinafter, preferred embodiments of the present invention will be described with reference to the accompanying drawings. In the following description of the present invention, a detailed description of known functions and configurations incorporated herein will be omitted when it may make the subject matter of the present invention rather unclear.

The invention relates to a use of sphingomyelin protecting skin from the sunlight, rapidly repairing physical injuries of the skin and inhibiting the skin aging.

When the skin is exposed to ultraviolet, an inflammatory response occurs within 3˜4 hours and collagen synthesis is increased. However, since activations of collagenase and elastinase are also increased, contents of collagen and elastin are decreased compared to the normal skin and thus wrinkles occur.

When the photoaging goes on, thickness of epidermis and dermis are decreased and keratinocyte and fibroblast are decreased. This results from a decrease of divisions of cells constituting the epidermis and the dermis. Sphingolipid participate in such changes. Meanwhile, the changes occurring due to the photoaging affect a dermal-epidermal junction, thereby decreasing support functions of the epidermis and the dermis. In addition, screening permeation function becomes also weak, so that noxious substances are delivered to the dermis. Further, deformed collagen, for example collagen bonded to sugar, and elastin are increased, thereby causing a hypofunction.

Under such circumstances, sphingomyelin acts as a skin protecting substance, thereby making it possible to activate the skin metabolism and thus to inhibit an occurrence of wrinkles.

Meanwhile, since sphingomyelin has an excellent effect of rapidly repairing the injured skin, it is effective in repairing the injured skin.

Sphingomyelin used in the invention may be extracted from milk or an egg, or brain tissues or erythrocytes of an animal.

The compound of the invention may be medicated in a manner of oral, parenteral, topical, percutaneous, intravenous, intramuscular, intraperitoneal or hypodermic administration. Dosage of active compound may be different depending on the subjects of treatment, specific diseases or pathological states to be treated, a severity of a disease or pathological state, an administration path and a judgment of a prescriber. A decision of dosage based on these factors is within a level of those skilled in the art. Typically, the dosage will be between 0.01 mg/kg·day and 2000 mg/kg·day. A preferred dosage will be between 0.5 mg/kg·day and 2.5 mg/kg·day.

The compound of the invention can be formulated into pharmaceutical composition together with a pharmaceutically acceptable carrier. A reference material (Remington's Pharmaceutical Sciences, latest edition, by E. W. Martin (Merck Publ. Co., Easton, Pa.)) discloses a typical carrier and a conventional manufacturing method of pharmaceutical composition, which can be used for manufacturing the composition of the invention. The composition of the invention can be medicated along with other compositions and procedures for treating a disease. For example, the composition of the invention may be medicated along with a radiotherapy or chemotherapy.

The pharmaceutical composition may be medicated in the form of a solid, semi-solid or liquid depending on an intended administration pattern. It may include a tablet, pill, capsule, suppository, small bag, granule, powder, cream, lotion, ointment, stick blaster, liquid solution, suspension, dispersion, emulsion and syrup, but is not limited to them. Active ingredients may be capsulated in liposome, fine particles or microcapsules.

A typical nontoxic carrier may include mannitol, lactose, starch, magnesium stearate, sodium saccharine, talc, cellulose, glucose, sucrose, dextrose, glycerol, magnesium carbonate, triglyceride, oil, solvent, sterile water and a pharmaceutical level of isotonic saline solution, but is not limited to them. Solid composition such as tablet, pill and granule, etc. may be conveniently coated. Typically, composition for intravenous administration is a solution in a sterile isotonic aqueous buffer and comprises a topical anesthetic for alleviating a pain in an injection region. If desired, a medicament may comprise a small amount of nontoxic ancillary substance such as a wetting agent, emulsifying agent, ph buffering agent, etc. The ancillary substance may include sodium acetate, sorbitan monolaurate, triethanolamine and triethanolamine oleate, but is not limited to them. The composition of the invention may comprise an excipient such as a stabilizer, an antioxidant, a bonding agent, a colorant, a cordial, an antiseptic and a thickening agent.

Preferably, the composition of the invention comprises sphingomyelin in an amount of 0.001˜99 wt. % of the total composition. When the content of shpingomyelin is below 0.001 wt. %, the skin protecting effect is insignificant. In addition, it is difficult to comprise sphingomyelin in an amount of 99 wt. % more due to other additives or impurities.

In order that this invention may be better understood, the following examples are set forth. These examples are for the purpose of illustration only and are not to be construed as limiting the scope of the invention in any manner.

### EXAMPLE 1

### A Photoaging Inhibitory Effect of Sphingomyelin (Animal Test)

8˜9 week old hairless mice were subject to 7 days of adaptation period. Then, mice exhibiting an increase in weight and no abnormality in general symptoms were selected. After that, UV B and UV A were irradiated to the selected mice by using an ultraviolet irradiator. The ultraviolet irradiation was carried out three times per a week for three months and ultraviolet dose was about 20 J/cm². After the ultraviolet irradiation, samples having sphingomyelin and no sphingomyelin were evenly applied to both regions of the back.

Test material was prepared by dissolving it in concentration of 0.5% in a medium in which 1,3-butyleneglycol, distilled water and ethanol were mixed in a ratio of 5:3:2, and then used.

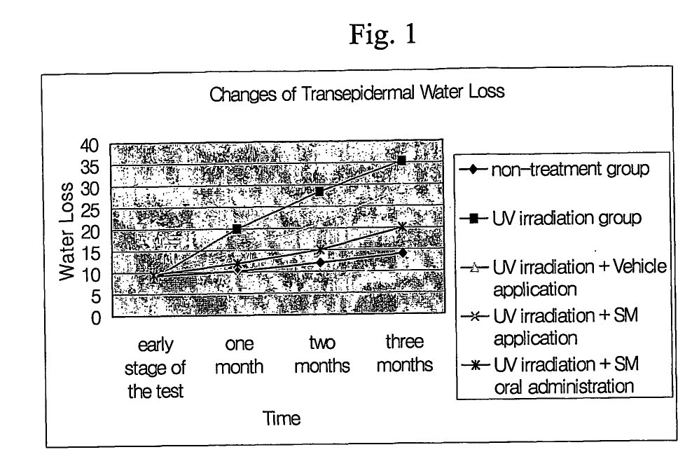

After three months from the completion of the test, measurement results of skin barrier function are shown in FIG. 1.

As shown in FIG. 1, when sphingomyelin was applied together with UV irradiation (UV irradiation+SM application), the skin barrier was not easily damaged and remained in a level of the early stage of the test as time went by, compared to the case that none was applied (UV irradiation group) or only medium was applied (UV irradiation+Vehicle application group). In addition, it was shown that the skin barrier protecting effect of sphingomyelin against ultraviolet was exhibited in the case of oral administration (UV irradiation+SM oral administration) as well as in the case of application.

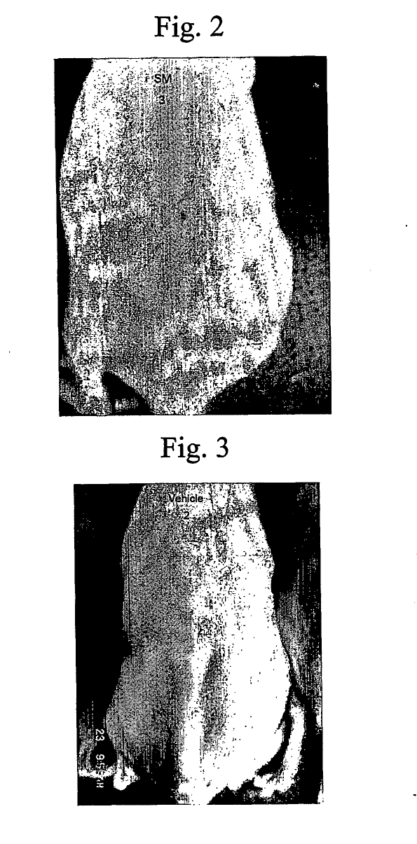

In order to measure a rat's state of skin after the completion of the test, it was taken a photograph of rat's skin. The results are shown in FIGS. 2 to 4.

FIG. 2 is a photograph showing a rat's state of skin in the case of UV irradiation+SM application, FIG. 3 is a photograph showing a rat's state of skin in the case of UV irradiation+Vehicle (medium) application, and FIG. 4 is a photograph showing a rat's state of skin when only UV is irradiated.

As shown in FIGS. 2 to 4, it can be seen that when only UV was irradiated or when UV was irradiated and the medium only was applied, the skin was extremely injured, compared to the case that the substrate containing sphingomyelin was applied after UV irradiation.

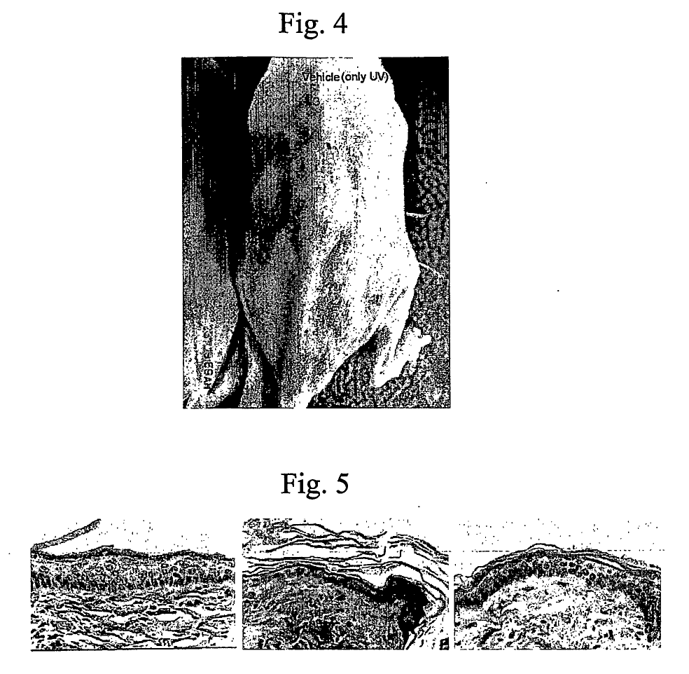

In addition, in order to measure a rat's state of skin after the completion of the test, it was carried out a histological test. The results are shown in FIG. 5.

FIG. 5 shows a skin tissue of a rat at the early stage of the test, a skin tissue of a rat irradiated with UV and a skin tissue of a rat applied by sphingomyelin after UV irradiation, from left. As shown in FIG. 5, in the case of only UV irradiation, the epidermis of the rat became very thinned and collagen seemed to be deformed since it seemed that synthesis of collagen was active, but an arrangement of collagens was irregular (middle). To the contrary, in the case of the experimental group applied by sphingomyelin, it can be seen that the epidermis and dermis thereof were similar to those of at the early stage of the test (right).

### Example 2

### A Wound Curing Effect of Sphingomyelin (Animal Test)

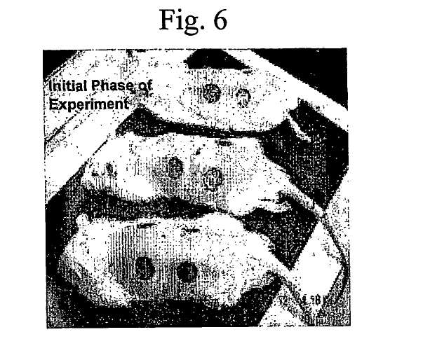

A wound curing test was carried out using a SD (Sprague-Dawley) male rat having the weight of 200 g. The SD rat was anesthetized with 5% chloral hydrate and then the back region thereof was hair-removed. After the hair-removal, skin in the back region was incised in a predetermined size and thus about 1.5 cm of an injury was made (FIG. 6). Samples used in the invention were sphingomyelin derived from milk and an egg and hydrogenated sphingomyelin thereof. Test material was prepared by dissolving it in concentration of 0.5% in a substrate in which 1,3-butyleneglycol, distilled water and ethanol were mixed in a ratio of 5:3:2, and then used. The test material was applied to the injured region two times every day and then results after 7 days and 11 days were decided. The results are shown in FIGS. 7 and 8.

FIG. 6 is a photograph showing an early stage of a test for examining a wound curing effect of sphingomyelin, FIG. 7 is a photograph showing a state after 7 days from a certain treatment, and FIG. 8 is a photograph showing a state after 11 days from the treatment.

In FIGS. 7 and 8, an upper left shows a substrate application group, an upper right shows a milk-derived sphingomyelin application group, a lower left shows an egg-derived sphingomyelin application group and a lower right shows an injury curing formulation (trademark: Fucidin) application group.

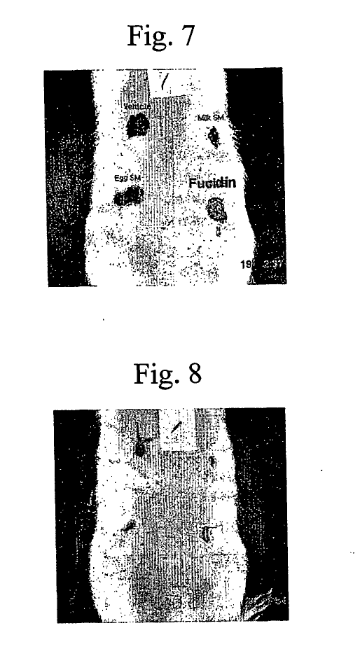

Meanwhile, FIGS. 9 and 10 illustrate a result of test for demonstrating a wound curing effect of hydrogenated sphingomyelin. Specifically, FIG. 9 is a photograph showing a state after 7 days from a treatment, and FIG. 10 is a photograph showing a state after 11 days from the treatment.

In FIGS. 9 and 10, upper left and right show substrate application groups, a lower left shows a milk-derived sphingomyelin application group, and a lower right shows an egg-derived sphingomyelin application group.

Each of biopsy regions was converted into grades by explaining the states of the repair numerically and then results thereof were decided.

5 points: no wound

4 points: very slight wound (barely discernible with naked eyes)

3 points: a repair phenomenon in the injured region, which can be observed with naked eyes

2 points: a wound repair is insufficiently observed

1 point: expansion of wound and occurrence of other edemas

Based on the above criteria, an average value of each experimental group is as follows.

total substrate application group (Vehicle)=1.7

milk-derived sphingomyelin application group (Milk SM)=2.4

egg-derived sphingomyelin application group (Egg SM)=2.8

injury curing formulation application group (Fucidin)=1.6

milk-derived and hydrogenated sphingomyelin application group (Milk HSM)=1.6

egg-derived and hydrogenated sphingomyelin application group (Egg HSM)=2.0

As it can be seen from the above results, both milk-derived and egg-derived sphingomyelins had an excellent effect. Hydrogenated sphingomyelin was less effective than non-hydrogenated sphingomyelin.

### EXAMPLE 3

### A Skin barrier Function Improving Effect of Sphingomyelin

In this Example, it was measured that a skin barrier function is improved by applying sphingomyelin and thus cutaneous disorders such as atopy and itchy skin are prevented and cured.

In this Example, a person's sebum film was artificially injured to increase an amount of water loss. After that, it was confirmed that there was a difference of repair degrees of water loss before and after taking sphingomyelin. It was also confirmed that how much skin wrinkles due to the water loss were improved by taking sphingomyelin.

A capsule containing sphingomyelin 50 mg, phosphatidylserine 10 mg and gamma-linoleic acid 12.5 mg was used as a test material. Two capsules per one time were taken two times (morning and evening) a day. That is, four capsules were taken per a day and a medication period was three weeks.

Tests were carried out for 18 males and 3 females and the testing method was as follows. An amount of water loss of skin was measured for an inner region of an arm using a TEWL meter TM210. A skin wound was induced by stripping off corneum with a tape many times and increasing a TEWL value (normally, around 6˜10) to 30 or more.

In other words, after inducing an injury of a skin barrier using a tape, repair aspects of TEWL values in the injured region before and after taking sphingomyelin were compared and thus a skin barrier restoring effect of sphingomyeling was confirmed.

Meanwhile, in order to confirm whether sphingomyelin had an improvement effect of skin wrinkles, a wrinkle improving effect was checked by observing wrinkles adjacent to eyes with naked eyes using a Charm View and comparing the wrinkles before and after taking sphingomyelin.

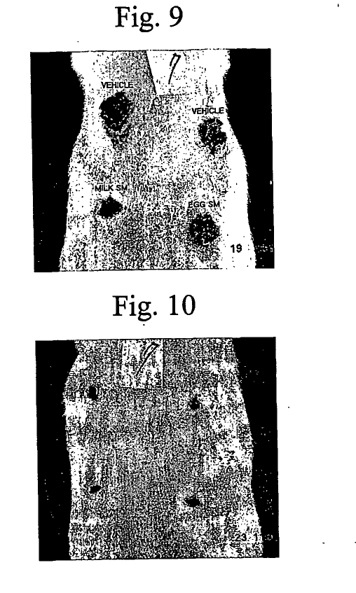

Skin barrier function in a normal state was checked by measuring TEWL in a forearm from 21 persons. A TEWL value was increased to 30 or more after tape stripping and then TEWL was measured every other day. TEWL in a normal state was measured together with a measurement of TEWL in an injured region because TEWL in a normal state can be different according to conditions of temperature, humidity and weather, etc. Meanwhile, a same test was carried out to measure a medication effect of sphingomyelin and TEWL of skin was measured while taking sphingomyelin two times a day. Results are shown in Table 3 and FIG. 11.

TABLE 3

| | Not-taking sphingomyelin | Taking sphingomyelin |
|---|---|---|
| days | (% TEWL) | (% TEWL) |
| 0 | 100 | 100 |
| 2 | 47.6 ± 18.9 | 43.2 ± 11.5 |
| 4 | 25.6 ± 13.3 | 23.1 ± 8.1 |
| 6 | 20.2 ± 8.9 | 14.8 ± 7* |

As shown in Table 3, when 2 and 4 days went by, there was no significant difference between when taking sphingomyelin and when not-taking sphingomyelin. However, it was confirmed that when taking sphingomyelin, the TEWL value was decreased compared to when not-taking sphingomyelin on six days.

Meanwhile, as shown in FIG. 11, variations of TEWL (Transepidermal water loss) average values can be seen when not taking sphingomyelin (before medication) and when taking sphingomyelin (after medication), after injuring sebum film of the skin. There was a significant difference in a TEWL decrease on six days when taking sphingomyelin (paired t-test. *p<0.055).

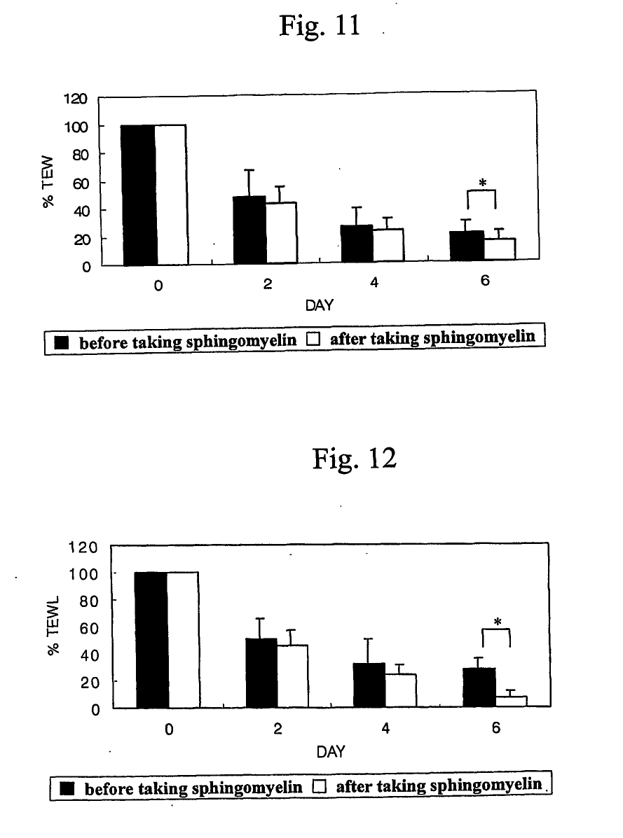

Meanwhile, a decrease effect of TEWL by sphingomyelin was tested for examinees who exhibited a high decrease rate of TEWL. The difference of decrease effects was further significantly shown in light of results of six examinees having exhibited a relatively high decrease rate of TEWL. The results are shown in Table 4 and FIG. 12.

TABLE 4

| | Not-taking sphingomyelin | Taking sphingomyelin |
|---|---|---|
| Days | (% TEWL) | (% TEWL) |
| 0 | 100 | 100 |
| 2 | 50.8 ± 15.2 | 45.7 ± 10.7 |
| 4 | 31.7 ± 18.8 | 23.7 ± 7.5 |
| 6 | 27.8 ± 8.1 | 7.4 ± 4.3* |

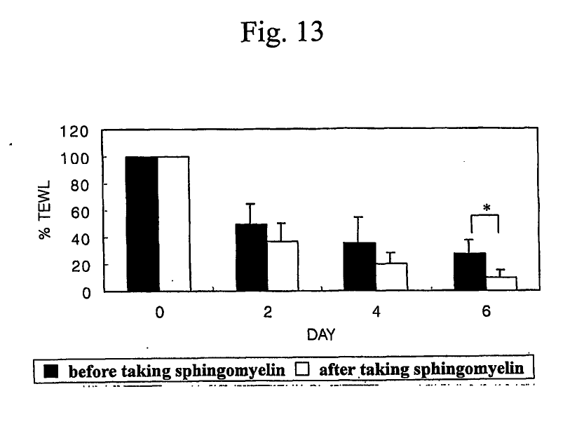

Meanwhile, a person, who had a relatively high value of TEWL in a normal region, i.e., who had more dry and atopic skin than other people because he or she had a weak skin barrier function, was selected and a test was carried out. Results are shown in Table 5 and FIG. 13.

TABLE 5

| | Not-taking sphingomyelin | Taking sphingomyelin |
|---|---|---|
| Days | (% TEWL) | (% TEWL) |
| 0 | 100 | 100 |
| 2 | 49.4 ± 15.3 | 36.9 ± 18.9 |
| 4 | 35.4 ± 18.9 | 19.8 ± 8 |
| 6 | 26.8 ± 10.5 | 9.7 ± 5.8* |

As shown in Table 5, there was no significant difference between a sphingomyelin taking group and a sphingomyelin not-taking group when 2 and 4 days went by. However, it can be seen that there was a significant difference of TEWL decreases between a sphingomyelin taking group and a sphingomyelin not-taking group on 6 days. This demonstrates that sphingomyelin has an excellent effect of repairing the damaged skin barrier function.

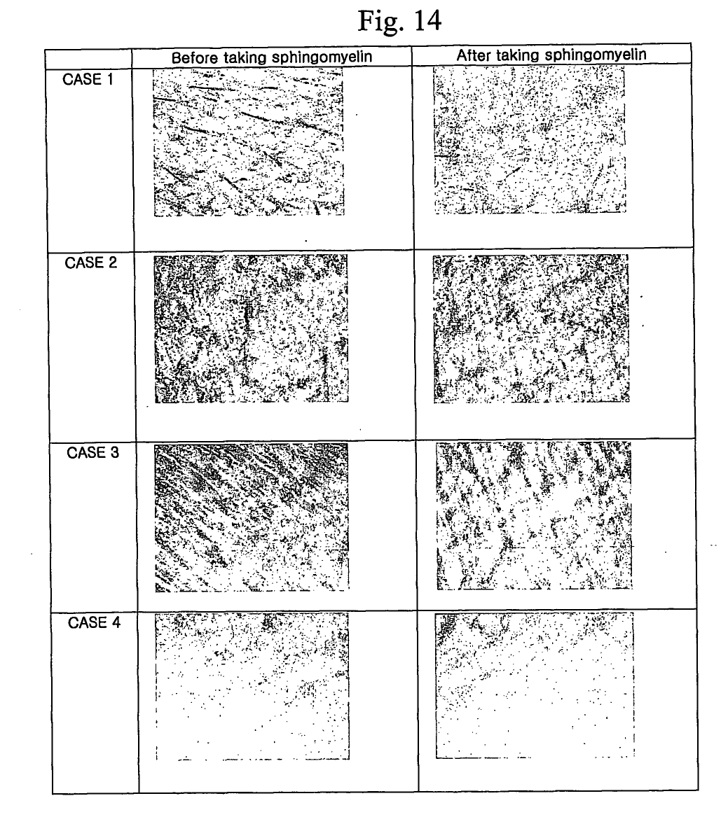

Meanwhile, in order to confirm whether sphingomyelin had an improvement effect of skin wrinkles, a wrinkle improving effect was checked by observing wrinkles adjacent to eyes with naked eyes using a Charm View and comparing the wrinkles before and after taking sphingomyelin. Four persons having a weak skin barrier function of the 21 examinees were selected and their skin states were measured before and after taking sphingomyelin. Results are shown in FIG. 14. As shown in FIG. 14, it was confirmed that sphingomyelin had a wrinkle improving effect.

The composition for protecting skin according to the invention can be prepared as follows.

### FORMULATION EXAMPLE 1

### Cream Containing 2% Sphingomyelin

TABLE 6

| Ingredients | Contents (wt. %) |
|---|---|
| Sphingomyelin | 2.0 |
| Propylene glycol | 20.0 |
| Stearyl alcohol | 6.5 |
| Cetyl alcohol | 3.5 |
| Sorbitan monostearate | 3.0 |
| Polysorbate 60 | 2.0 |
| Isopropyl myristate | 1.0 |
| Anhydrous sodium sulfite | 0.2 |
| Polysorbate 80 | 0.1 |
| Purified water | 61.7 |

Stearyl alcohol, cetyl alcohol, sorbitan monostearate and isopropyl myristate were introduced into a double-walled vessel and then heated until the mixture was completely dissolved. The mixture was added to a separately prepared mixture of purified water, propylene glycol and polysorbate 60 while using a homogenizer for liquid at 70˜75° C. The resulting emulsion was continuously mixed and cooled to below 25° C. A solution of sphingomyelin, polysorbate 80 and purified water and an anhydrous sodium sulfite solution in purified water were then continuously mixed and added to the emulsion. Cream was homogenized and filled into a proper tube.

### FORMULATION EXAMPLE 2

### Topical Gel Containing 2% Sphingomyelin

TABLE 7

| Ingredients | Contents (wt. %) |
|---|---|
| Sphingomyelin | 2.0 |
| Propylene glycol | 4.0 |
| Hydroxypropyl beta-cyclodextrin | 25.0 |
| Ethyl alcohol 95% (v/v) | 4.0 |
| Carrageenan PJ | 1.0 |
| Purified water | To 100 |

An appropriate amount of hydrochloric acid was added to the mixture to give a solution. An appropriate amount of sodium hydroxide was added to the solution to adjust the pH of the solution to 6.0. An appropriate amount of purified water was added to the solution to give 100 mg of the solution.

To a solution of hydroxypropyl beta-cyclodextrin in purified water was added sphingomyelin with stirring. Hydrochloric acid was added to the mixture to give a solution and then sodium hydroxide was added the solution to adjust the pH to 6.0. This solution was added to a dispersion of carrageenan PJ in propylene glycol with mixing. The mixture was heated to 50° C. with slowly mixing, added with ethyl alcohol and then cooled to about 35° C. The remaining purified water was added and then the mixture was mixed until the mixture was homogenized.

### FORMULATION EXAMPLE 3

### Topical Cream Containing 2% Sphingomyelin

TABLE 8

| Ingredients | Contents (wt. %) |
|---|---|
| Sphingomyelin | 2.0 |
| Hydroxypropyl beta-cyclodextrin | 20.0 |
| Stearyl alcohol | 2.5 |
| Cetyl alcohol | 2.5 |
| Mineral oil | 11.0 |
| Glycerol monostearate | 2.5 |
| Glycerol | 5.0 |
| Sorbate 60 | 2.0 |
| Polysorbate 60 | 3.5 |
| Purified water | To 100 |

An appropriate amount of hydrochloric acid was added to the mixture to give a solution. An appropriate amount of sodium hydroxide was added to the solution to adjust the pH of the solution to 6.0. An appropriate amount of purified water was added to the solution to give 100 mg of the solution.

To a solution of hydroxypropyl beta-cyclodextrin in purified water was added sphingomyelin with stirring. Hydrochloric acid was added to the mixture to give a solution and then sodium hydroxide was added to the solution to adjust the pH of the solution to 6.0. Glycerol and polysorbate 60 were added to the mixture with stirring and then the mixture was heated to 70° C. The resulting mixture was added to a mixture of mineral oil, stearyl alcohol, cetyl alcohol, stearyl monostearate and sorbate 60 at 70° C. with slowly mixing. Then, the mixture was cooled to below 25° C. The remaining purified water was added and then the mixture was mixed until the mixture was homogenized.

### FORMULATION EXAMPLE 4

### Liposome Formulation Containing 2% Sphingomyelin

TABLE 9

| Ingredients | Contents (wt. %) |
|---|---|
| Sphingomyelin | 2.0 |
| Phosphatidylcholine | 30.0 |
| Cholesterol | 5.0 |
| Ethyl alcohol | 10.0 |
| Methyl paraffin | 0.15 |
| Propyl paraffin | 0.02 |
| Disodium edetate | 0.15 |
| NaCl | 0.4 |
| hydroxypropylmethylcellulose | 1.2 |
| Purified water | To 100 |

Purified water was added to give 100 g of a solution.

A mixture of sphingomyelin, phosphatidylcholine, cholesterol and ethyl alcohol was stirred and heated at 55˜60° C. to give a solution. The solution was added to a solution of methyl paraffin, propyl paraffin, disodium edetate and NaCl in purified water with homogenizing. Hydroxypropylmethylcellulose in purified water was added, and then mixed continuously until swelling.

### FORMULATION EXAMPLE 5

### Liposome Formulation Containing 2% Sphingomyelin

TABLE 10

| Ingredients | Contents (wt. %) |
|---|---|
| Sphingomyelin | 2.0 |
| Phosphatidylcholine | 10.0 |
| Cholesterol | 1.0 |
| Ethyl alcohol | 7.5 |
| Hydroxypropylmethylcellulose | 1.5 |
| Purified water | To 100 |

Sodium hydroxide (1N) was added to adjust the pH to 5.0.

Purified water was added to give 100 g of a solution.

A mixture of phosphatidylcholine and cholesterol in ethyl alcohol was stirred and heated at 40° C. to give a solution. Sphingomyelin was dissolved in purified water with mixing and heating at 40° C. To the aqueous solution was slowly added alcoholic solution with homogenizing for 10 min. Hydroxypropylmethylcellulose in purified water was added and then mixed until swelling. The resulting solution was adjusted to pH 5.0 by adding 1 N sodium hydroxide and diluted with the remaining purified water.

### FORMULATION EXAMPLE 6

### Sphingomyelin Nanodispersion

(1) Sphingomyelin Nanodispersion Pre-Phase

TABLE 11

| Ingredients | Contents (wt. %) |
|---|---|
| Sphingomyelin | 36.6% |
| Phosphatidylcholine | 9.0% |
| Polysorbate 80 | 34.0% |
| Ethyl alcohol | 7.4% |
| Myglyol 812 | 13.0% |

Myglyol 812, sphingomyelin and polysorbate 80 were mixed. Phosphatidylcholine dissolved in ethanol was added to the mixture to give homogeneous clear liquid.

(2) Sphingomyelin Nanodispersion Aqueous Phase

TABLE 12

| Ingredients | Contents (wt. %) |
|---|---|
| Sphingomyelin | 2.0 |
| Phosphatidylcholine | 0.49 |
| Polysorbate 80 | 1.86 |
| Ethyl alcohol | 0.63 |
| Myglyol 812 | 0.71 |
| Purified water | To 100.0 |

Aqueous phase containing sphingomyelin (for example, 94.54 g) was placed in a vessel with stirring at 50° C. The liquid nanodispersion pre-phase (for example, 5.46 g) was added to the aqueous phase with stirring.

### FORMULATION EXAMPLE 7

### Medical Ointment Base Formulations

TABLE 13

| Ingredients | Contents (wt. %) |
|---|---|
| Lanolin alcohol | 1 |
| Stearyl alcohol | 2 |
| Ceteareth-20 | 2 |
| Perlatum | 84.5 |
| Lecithin | 1.5 |
| Caprylic/Capric triglyceride | 2 |
| PEG20 Corn glycerides | 5 |
| sphingomyelin | 2 |

### FORMULATION EXAMPLE 8

### Cosmetic Cream Formulation

TABLE 14

| | INCI Name | Contents (wt. %) |
|---|---|---|
| Aqueous phase | Disodium EDTA | 0.020 |
| | Glycerine | 4.000 |
| | Butylene glycol | 2.000 |
| | Xanthan gum | 0.030 |
| | Triethanolamine | 0.200 |
| | Di-water | To 100 |
| | Carbomer | 0.1 |
| Oil phase | Stearic acid | 1.800 |
| | Glyceryl stearate | 1.000 |
| | PEG-100 stearate | |
| | Setearyl alcohol | 2.000 |
| | Glyceryl stearate | 2.000 |
| | Sorbitan sesquioleate | 0.300 |
| | Polysorbate 60 | 1.200 |
| | Mineral oil | 6.000 |
| | Isopropyl myristate | 1.500 |
| | Cetyl octanoate | 1.000 |
| | Sphingomyelin | 2.000 |
| | Dimethicone | 0.400 |
| | Preservative | Q.S. |

Aqueous phase and oil phase were heated to 75° C., respectively.

After checking complete dissolution of the aqueous phase and the oil phase, the aqueous phase was introduced into a major oven.

The oil phase was slowly introduced in the major oven, stirred using homomixer (3,500 rpm) and peddlemixer (30 rpm) for 3 min, and then was cooled.

According to the invention as described above, it can be prevented and repaired that as the aging goes on, the skin becomes thin, the generation of collagen is decreased, a wrinkle occurs, the skin loses its elasticity, abnormal blood vessels are developed and pigmentation such as dark spots occurs. In addition, it can be prevented and cured that when the skin is exposed to the sunlight, collagen and elastic fibers of the skin are injured and thus the skin loses its elasticity and a wrinkle occurs. Further, it is possible to cure or improve atopic skin having a weak skin barrier function by improving the skin barrier homeostasis, and to rapidly repair the skin barrier damaged by ultraviolet, etc. Additionally, the invention has effects of improving itchy skin symptoms, softening the skin and preventing the generation of rough and scaly skin. A skin protecting effect is also provided by repairing the skin wound.

While the invention has been shown and described with reference to certain preferred embodiments thereof, it will be understood by those skilled in the art that various changes in form and details may be made therein without departing from the spirit and scope of the invention as defined by the appended claims.

What is claimed is:

1. A composition for protecting skin comprising sphingomyelin as an active ingredient.

2. Use of the composition according to claim 1 for inhibiting a skin aging.

3. Use of the composition according to claim 1 for inhibiting a skin photoaging.

4. Use of the composition according to claim 1 for curing a wound.

5. Use of the composition according to claim 1 for improving a skin barrier function.

6. The use according to claim 5, wherein the use is one for rapidly repairing a damaged skin barrier.

7. Use of the composition according to claim 1 for treating an atopic skin.

8. Use of the composition according to claim 1 for improving an itchy skin.

9. Use of the composition according to claim 1 for improving a softness of skin.

10. Use of the composition according to claim 1 for preventing rough and scaly skin formation.

11. The composition according to claim 1, wherein the sphingomyelin is derived from milk or an egg.

12. The composition according to claim 1, wherein the sphingomyelin is hydrogenated sphingomyelin.

13. The composition according to claim 1, wherein the composition is a composition for oral administration or topical application.
